(19) United States
(12) Patent Application Publication
(10) Pub. No.: US 2004/0093066 A1
(43) Pub. Date: May 13, 2004

(54) Balloon expandable stent

(76) Inventor: Jonathan P. Durcan, Temecula, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/255,479

(22) Filed: Sep. 26, 2002

(51) Int. Cl. A61F 2/06

(52) U.S. Cl. 623/1.15

(57) ABSTRACT

A balloon expandable intravascular stent assembly for implantation in a body vessel, such as a coronary artery, is designed to provide stent coverage beyond the balloon working length and minimize damage to the vessel wall during balloon inflation. The stent consists of radially expandable cylindrical rings generally aligned on a common longitudinal stent axis and either directly connected or interconnected by one or more interconnecting links. The stent includes a distal section, proximal section, and center section. The rings within the distal section and proximal section may be configured with a nickel-titanium alloy and may include tabs which extend away from the center section.

### BACKGROUND OF THE INVENTION

The present invention relates generally to expandable endoprosthesis devices, generally called stents, which are adapted to be implanted into a patient's body lumen, such as a blood vessel, to maintain the patency thereof, and more particularly to balloon expandable stents configured to provide coverage area beyond the balloon working length and minimize damage to the vessel wall during balloon inflation.

Stents are particularly useful in the treatment and repair of blood vessels after a stenosis has been compressed by percutaneous transluminal coronary angioplasty (PTCA), percutaneous transluminal angioplasty (PTA), or removed by atherectomy or other means, to help improve the results of the procedure and reduce the possibility of restenosis. Stents also can be used to provide primary compression to a stenosis in cases in which no initial PTCA or PTA procedure is performed. While stents are most often used in the procedures mentioned above, they also can be implanted on another body lumen such as the carotid arteries, peripheral vessels, urethra, esophagus and bile duct.

In typical PTCA procedures, a guiding catheter or sheath is percutaneously introduced into the cardiovascular system of a patient through the femoral arteries and advanced through the vasculature until the distal end of the guiding catheter is in the aorta. A guidewire and a dilatation catheter having a balloon on the distal end are introduced through the guiding catheter with the guidewire sliding within the dilatation catheter. The guidewire is first advanced out of the guiding catheter into the patient's vasculature and is directed across the arterial lesion. The dilatation catheter is subsequently advanced over the previously advanced guidewire until the dilatation balloon is properly positioned across the arterial lesion. Once in position across the lesion, the expandable balloon is inflated to a predetermined size with a radiopaque liquid at relatively high pressure to displace the atherosclerotic plaque of the lesion against the inside of the artery wall and thereby dilate the lumen of the artery. The balloon is then deflated to a small profile so that the dilatation catheter can be withdrawn from the patient's vasculature and the blood flow resumed through the dilated artery. As should be appreciated by those skilled in the art, while the abovedescribed procedure is typical, it is not the only method used in angioplasty.

In angioplasty procedures of the kind referenced above, abrupt reclosure may occur or restenosis of the artery may develop over time, which may require another angioplasty procedure, a surgical bypass operation, or some other method of repairing or strengthening the area. To reduce the likelihood of the occurrence of abrupt reclosure and to strengthen the area, a physician can implant an intravascular prosthesis for maintaining vascular patency, commonly known as a stent, inside the artery across the lesion. Stents are generally cylindrically shaped devices which function to hold open and sometimes expand a segment of a blood vessel or other arterial lumen, such as A coronary artery. Stents are usually delivered in a compressed condition to the target location and then are deployed into an expanded condition to support the vessel and help maintain it in an open position. The stent is usually crimped tightly onto a delivery catheter and transported in its delivery diameter through the patient's vasculature. The stent is expandable upon application of a controlled force, often through the inflation of the balloon portion of the delivery catheter, which expands the compressed stent to a larger diameter to be left in place within the artery at the target location. The stent also may be of the self-expanding type formed from, for example, shape memory metals or super-elastic nickel-titanum (NiTi) alloys, which will automatically expand from a compressed state when the stent is advanced out of the distal end of the delivery catheter into the body lumen.

The above described, non-surgical interventional procedures, when successful, avoid the necessity for major surgical operations. However, a danger which is always present during the balloon inflation procedure in a balloon inflatable stent is the potential for damaging the vessel wall with the balloon outside the area of the vessel wall contacted by the stent. During deployment of a stent, the balloon must completely expand the stent against the vessel wall. The portion of the balloon which expands the stent is commonly referred to as the balloon working length. To completely expand the stent from its proximal end to its distal end, the balloon working length must be slightly longer than the expanded stent. Therefore, when the balloon is completely expanded, portions of the balloon come into contact with the vessel wall outside the distal and proximal ends of the expanded stent. The resulting balloon contact with the vessel wall may damage the wall.

Another problem area has been providing stent coverage beyond the balloon working length used to expand the stent. The balloon working length must be long enough to ensure that the outermost rings are completely expanded. Prior art stent vessel coverage areas are consequently limited by balloon working length.

What has been needed is a balloon inflatable stent which has a high degree of flexibility so that it can be readily advanced through tortuous passageways and radially expanded over a wide range of diameters while incorporating a higher degree of safety when expanded and a higher vessel wall coverage area. A stent is needed that covers the vessel wall over an area that is longer than the working length of the balloon. The present invention satisfies these needs.

### SUMMARY OF THE INVENTION

The present invention is directed to a balloon inflatable intravascular stent assembly for implantation in a body vessel, such as a coronary artery. The stent is designed to provide coverage beyond the balloon working length and to minimize damage to the vessel wall during balloon inflation.

The stent assembly embodying features of the invention can be readily delivered to the desired body lumen, such as a coronary artery (peripheral vessels, bile ducts, etc.), by mounting the stent assembly on an expandable member of a delivery catheter, for example a balloon, and advancing the catheter and stent assembly through the body lumen to the target site. Generally, the stent is crimped onto the balloon portion of the catheter so that the stent assembly does not move longitudinally relative to the balloon portion of the catheter during delivery through the arteries, and during expansion of the stent at the target site. The stent is relatively flexible along its longitudinal axis to facilitate delivery through tortuous body lumens yet is stiff and stable enough radially in an expanded condition to maintain the patency of a body lumen such as an artery when implanted therein.

In one embodiment, the stent of the invention includes a series of cylindrical rings formed with undulations and located within a distal section, center section, and proximal section of the stent. The rings located in the center section are formed from a plastically deformable metal and may have larger longitudinal lengths than the rings in the distal section and proximal section which may be formed from a self-expanding material such as nickel-titanium. Links are incorporated to connect the cylindrical rings within the center section together while the rings within the distal section and proximal section may be directly connected to adjacent rings within the center section.

In another embodiment, the stent of the present invention includes a series of cylindrical rings with undulations and also located within a distal section, center section, and proximal section of the stent. The rings located in the center section may have smaller longitudinal lengths than the rings in the distal section and proximal section. The rings within the distal section and proximal section may incorporate substantially linear tabs on either the peaks or the valleys of the undulations. The tabs may be aligned along the longitudinal axis of the stent and extend away from the center section of the stent. Adjacent rings may be connected through a series of links.

The resulting stent structures are a series of radially expandable cylindrical rings which are configured so that the stent provides coverage beyond the balloon working length and minimizes damage to the vessel wall during balloon inflation while maintaining the longitudinal flexibility of the stent both when being negotiated through the body lumens in their unexpanded state and when expanded into position.

The additional stent coverage area is provided by either the self-expanding rings located in the distal section and proximal section or the tabs incorporated into the rings located in the distal section and proximal section which extend away from the center section of the stent and along the longitudinal axis of the stent. Because neither the self-expanding rings nor the tabs portion of the rings of the stent require balloon inflation, the balloon working length can generally be held to the same length as for a conventional stent. This increase in stent coverage area of the present invention provides more area on which to deliver drugs and support a vessel and increases safety by minimizing balloon contact with the vessel wall.

Within the cylindrical rings, undulations allow for an even expansion around the circumference by accounting for the relative differences in stress created by the radial expansion of the cylindrical rings. Each of the individual cylindrical rings may rotate slightly relative to their adjacent cylindrical rings without significant deformation, cumulatively providing a stent flexible along its length and about its longitudinal axis, but which is still very stable in the radial direction in order to resist collapse after expansion.

Each of the embodiments of the invention can be readily delivered to the desired luminal location by mounting them on an expandable member of a delivery catheter, for example a balloon, and passing the catheter-stent assembly through the body lumen to the implantation site. A variety of means for securing the stents to the expandable member on the catheter for delivery to the desired location are available. It is presently preferred to crimp the stent onto the unexpanded balloon. Other means to secure the stent to the balloon include providing ridges or collars on the inflatable member to restrain lateral movement, using bioabsorbable temporary adhesives, or a retractable sheath to cover the stent during delivery through a body lumen.

The presently preferred structures for the expandable cylindrical rings which form the stents of the present invention generally have a plurality of circumferential undulations containing a plurality of alternating peaks and valleys. The peaks and valleys are formed in generally U- and W-shaped patterns alternately aligned along the longitudinal axis.

While the cylindrical rings, links, and tabs incorporated into cylindrical rings are generally not separate structures, they have been conveniently referred to as rings, links, and tabs for ease of identification. Further, the cylindrical rings can be thought of as comprising a series of U- and W-shaped structures in a repeating pattern along with tabs in certain embodiments. While the cylindrical rings are not divided up or segmented into U's and W's, the pattern of cylindrical rings resemble such configuration. The U's and W's promote flexibility in the stent primarily by flexing and may tip radially outwardly as the stent is delivered through a tortuous vessel. The tabs at the stent ends are not designed to tip radially outwardly and it is not a desirable feature at the stent ends because it may irritate the vessel wall.

The undulations of the cylindrical rings can have different degrees of curvature and angles of adjacent peaks and valleys to compensate for the expansive properties of the peaks and valleys. The cylindrical rings of the stents within the center section are plastically deformed when expanded so that the stent will remain in the expanded condition and therefore they must be sufficiently rigid when expanded to prevent the collapse thereof in use.

The rings located in the distal section and proximal section in one embodiment include one distal ring and one proximal ring, respectively. These rings are formed from self-expanding, super-elastic nickel-titanium (NiTi) alloys and the expansion of the rings occurs when the stress of compression is removed. This allows the phase transformation from martensite back to austenite to occur, and as a result the stent expands. Because the distal ring and proximal ring are directly attached to the rings within the center section they will remain in compressed form until the stent is expanded by a balloon.

After the stents are expanded some of the peaks and/or valleys may, but not necessarily, tip outwardly and embed in the vessel wall. Thus, after expansion, the stents may not have a smooth outer wall surface, rather they have small projections which embed in the vessel wall and aid in retaining the stents in place in the vessel.

The links which interconnect adjacent cylindrical rings and the tabs which are incorporated within the distal rings and the proximal rings and which extend outward from the center section can have cross-sections similar to the cross-sections of the undulating components of the cylindrical rings. The links may be formed in a unitary structure with the expandable cylindrical rings incorporating the tabs formed from the same intermediate product, such as a tubular element, or they may be formed independently and mechanically secured between the expandable cylindrical rings. The links and tabs may be formed substantially linearly or with a plurality of undulations.

Preferably, the number, shape and location of the links and tabs can be varied in order to develop the desired coverage area and longitudinal flexibility. These properties are important to minimize alteration of the natural physiology of the body lumen into which the stent is implanted and to maintain the compliance of the body lumen which is internally supported by the stent. Generally, the greater the longitudinal flexibility of the stents, the easier and the more safely they can be delivered to the implantation site, especially where the implantation site is on a curved section of a body lumen, such as a coronary artery or a peripheral blood vessel, and especially saphenous veins and larger vessels.

The stent may be formed from a tube by laser cutting the pattern of cylindrical rings, links, and tabs in the tube, by individually forming wire rings and laser welding them together, and by laser cutting a flat metal sheet in the pattern of the cylindrical rings, cylindrical rings incorporating tabs and links and then rolling the pattern into the shape of the tubular stent and providing a longitudinal weld to form the stent. The stent of the invention also can be coated with a drug or therapeutic agent. Further, it is well known that the stent (when made from a metal) may require a primer material coating such as a polymer to provide a substrate on which a drug or therapeutic agent is coated since some drugs and therapeutic agents do not readily adhere to a metallic surface. The drug or therapeutic agent can be combined with a coating or other medium used for controlled release rates of the drug or therapeutic agent.

Other features and advantages of the present invention will become more apparent from the following detailed description of the invention, when taken in conjunction with the accompanying exemplary drawings.

### BRIEF DESCRIPTION OF THE DRAWINGS

FIG. 1 is an elevational view, partially in section, of a stent embodying features of the invention which is mounted on a delivery catheter and disposed within a damaged artery.

FIG. 2 is an elevational view, partially in section, similar to that shown in FIG. 1 wherein the stent is expanded within a damaged or diseased artery.

FIG. 3 is an elevational view, partially in section, depicting the expanded stent within the artery after withdrawal of the delivery catheter.

FIG. 4 is a perspective view of the stent of FIG. 3 in its expanded state depicting the serpentine pattern along the peaks and valleys that form the cylindrical rings.

FIG. 5 is a plan view of a flattened section of one embodiment of a stent of the invention.

FIG. 6 is a partial elevational view of one embodiment of a stent of the invention in the compressed state.

FIG. 7 is a partial elevational view of the stent shown in FIG. 6 in the expanded state.

FIG. 8 is a plan view of a flattened section of one embodiment of a stent of the invention.

FIG. 9 is a partial elevational view of one embodiment of a stent of the invention in the compressed state.

FIG. 10 is a partial elevational view of the stent shown in FIG. 9 in the expanded state.

### DETAILED DESCRIPTION OF THE PREFERRED EMBODIMENTS

Before describing in detail an exemplary embodiment of a balloon expandable stent in accordance with the present invention, it is instructive to briefly describe a typical stent implantation procedure and the vascular conditions which are typically treated with stents.

Turning to the drawings, FIG. 1 depicts a metallic stent 10 incorporating features of the invention mounted on a catheter assembly 12 which is used to deliver the stent and implant it in a body lumen, such as a coronary artery, peripheral artery, or other vessel or lumen within the body. The stent as shown in FIG. 2 generally includes a distal section 21, center section 23, and proximal section 25. Two sets of rings include a first set with a distal ring 13a located within the distal section and proximal ring 13b located within the proximal section and a second set with a series of center rings 11 located within the center section. The rings 11,13a,13b are generally radially expandable, disposed coaxially and interconnected by straight links 15 disposed between adjacent cylindrical rings. The catheter assembly shown in FIG. 1 includes a catheter shaft 13 which has a proximal end 14 and a distal end 16. The catheter assembly is configured to advance through the patient's vascular system by advancing over a guide wire by any of the well known methods of an over the wire (OTW) system (not shown) or a well known rapid exchange (RX) catheter system, such as the one shown in FIG. 1.

Catheter assembly 12 as depicted in FIG. 1 is of the well known rapid exchange type which includes an RX port 20 where the guide wire 18 will exit the catheter. The distal end of the guide wire exits the catheter distal end 16 so that the catheter advances along the guide wire on a section of the catheter between the RX port and the catheter distal end. As is known in the art, the guide wire lumen which receives the guide wire is sized for receiving various diameter guide wires to suit a particular application. The stent is mounted on the expandable member 22 (balloon) and is crimped tightly thereon so that the stent and expandable member present a low profile diameter for delivery through the arteries of a patient.

As shown in FIG. 1, a partial cross-section of an artery 24 is shown that has been previously treated by an angioplasty or other repair procedure. The stent assembly 10 of the present invention is used to repair a diseased or damaged arterial wall which may include a dissection, or a flap which are sometimes found in the coronary arteries, peripheral arteries and other vessels.

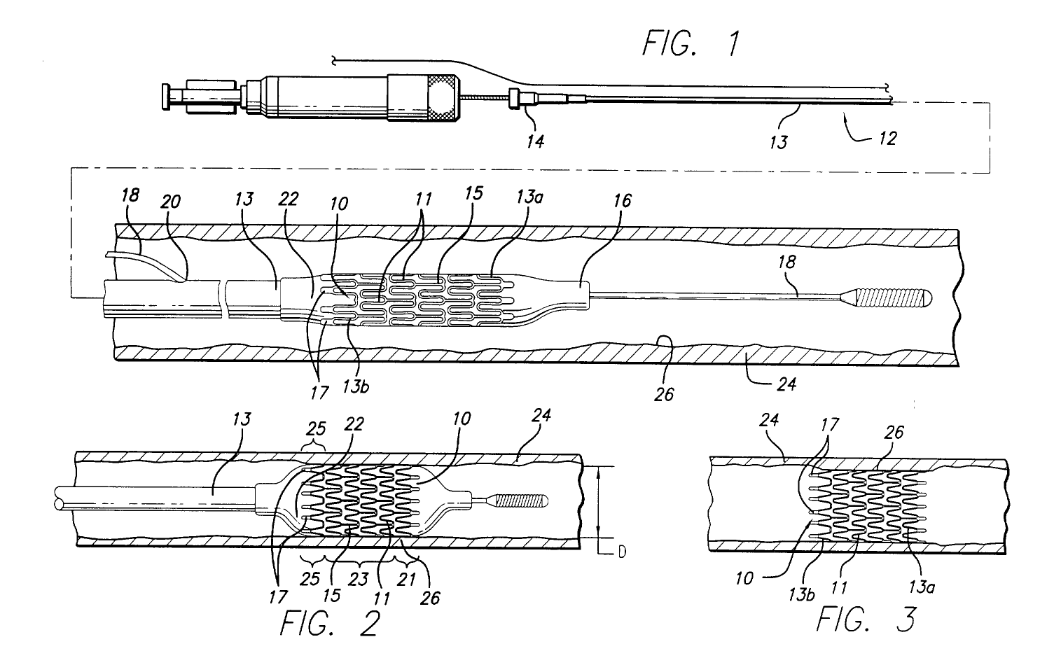

In a typical procedure to implant stent assembly 10, the guide wire 18 is advanced through the patient's vascular system by well known methods so that the distal end of the guide wire is advanced past the plaque or diseased area 26. Prior to implanting the stent assembly, the cardiologist may wish to perform an angioplasty procedure or other procedure (i.e., atherectomy) in order to open the vessel and remodel the diseased area. Thereafter, the stent delivery catheter assembly 12 is advanced over the guide wire so that the stent assembly is positioned in the target area. The expandable member or balloon 22 is inflated by well known means so that it expands radially outwardly and in turn expands the stent assembly radially outwardly until the stent assembly is apposed to the vessel wall. The balloon is then deflated and the catheter withdrawn from the patient's vascular system. The guide wire typically is left in the lumen for post-dilatation procedures, if any, and subsequently is withdrawn from the patient's vascular system. As depicted in FIG. 2, the balloon is fully inflated with the stent expanded and pressed against the vessel wall, and in FIG. 3, the implanted stent remains in the vessel after the balloon has been deflated and the catheter assembly and guide wire have been withdrawn from the patient.

The stent 10 serves to hold open the artery 24 after the catheter is withdrawn, as illustrated by FIG. 3. Due to the formation of the stent from an elongated tubular member, the undulating components of the stent are relatively flat in transverse cross-section, so that when the stent is expanded, it is pressed into the wall of the artery and as a result does not interfere with the blood flow through the artery. The stent is pressed into the wall of the artery and will eventually be covered with endothelial cell growth which further minimizes blood flow interference. The rings 11,13a,13b, and links 15 of the stent will eventually become endothelialized. It is this endothelialization and subsequent neointimal growth that will integrate the device into the stented portion of the artery. The undulating portions of the stent provide good tacking characteristics to prevent stent movement within the artery. Furthermore, the closely spaced cylindrical rings at regular intervals provide uniform support for the wall of the artery, and consequently are well adapted to tack up and hold in place small flaps or dissections in the wall of the artery.

The stent patterns shown in FIGS. 1-3 are for illustration purposes only and can vary in size and shape to accommodate different vessels or body lumens. Further, the stent 10 is of a type that can be used in accordance with the present invention.

The links 15 which interconnect adjacent cylindrical rings 11,13a,13b and the tabs 17 may have cross-sections similar to the cross-sections of the undulating components of the expandable cylindrical rings. In one embodiment, all of the links are joined at either the peaks or the valleys of the undulating structure of adjacent cylindrical rings. In this manner there is little or no shortening of the stent assembly upon expansion.

The number and location of the links 15 connecting the rings 11,13a,13b together can be varied in order to vary the desired longitudinal and flexural flexibility in the stent assembly structure both in the unexpanded as well as the expanded condition. These properties are important to minimize alteration of the natural physiology of the body lumen into which the stent assembly is implanted and to maintain the compliance of the body lumen which is internally supported by the stent assembly. Generally, the greater the longitudinal and flexural flexibility of the stent assembly, the easier and the more safely it can be delivered to the target site. Similarly, the number and location of the tabs 17 within the rings can be varied in order to vary the desired coverage area.

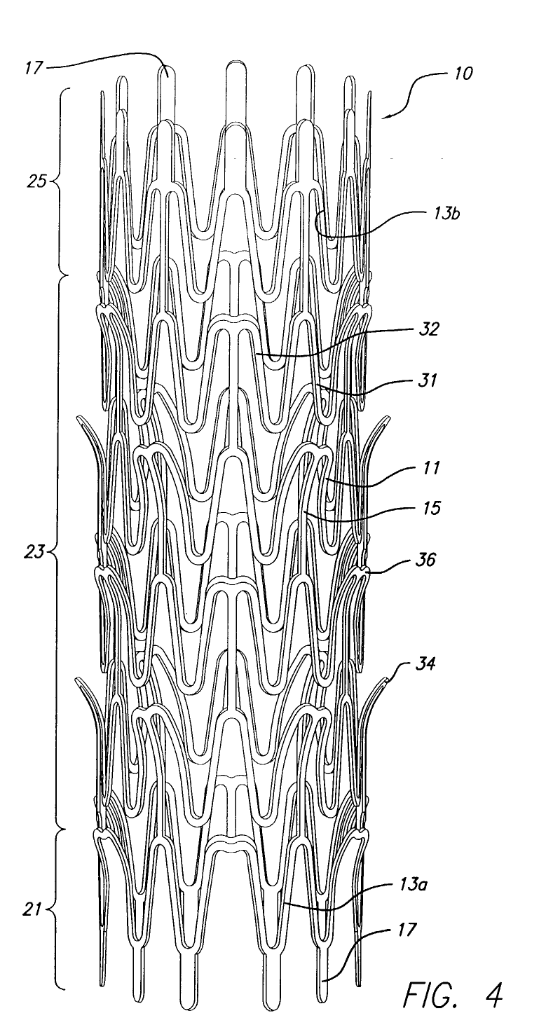

With reference to FIG. 4, the stent 10 includes cylindrical rings 11,13a, 13b in the form of undulating portions, the distal ring and proximal ring also including tabs 17. The undulating portion is made up of a plurality of U-shaped members 31 and W-shaped members 32 having radii that more evenly distribute expansion forces over the various members. After the cylindrical rings have been radially expanded, outwardly projecting edges 34,36 may be formed. That is, during radial expansion some of the U- and W-shaped members may tip radially outwardly thereby forming outwardly projecting edges. These outwardly projecting edges can provide for a roughened outer wall surface of the stent and assist in implanting the stent in the vascular wall by embedding into the vascular wall. In other words, the outwardly projecting edges may embed into the vascular wall, for example arterial vessel 24, as depicted in FIG. 3. Depending upon the dimensions of the stent and the thickness of the various members making up the serpentine pattern, any of the U- or W-shaped members can tip radially outwardly to form the projecting edges. The tabs 17 are not configured to tip outwardly because of potential damage to the vessel wall.

Cylindrical rings 11,13a,13b can be nested such that adjacent rings slightly overlap in the longitudinal direction so that one ring is slightly nested within the next ring and so on. The degree of nesting can be dictated primarily by the length of each cylindrical ring, the number of undulations in the rings, the thickness of the rings, and the radius of curvature, all in conjunction with the crimped or delivery diameter of the stent. If the rings are substantially nested one within the other, it may be difficult to crimp the stent to an appropriate delivery diameter without the various struts overlapping. It is also contemplated that the rings may be slightly nested even after the stent is expanded, which enhances vessel wall coverage. In some circumstances, it may not be desirable to nest one ring within the other, which is also contemplated by the invention.

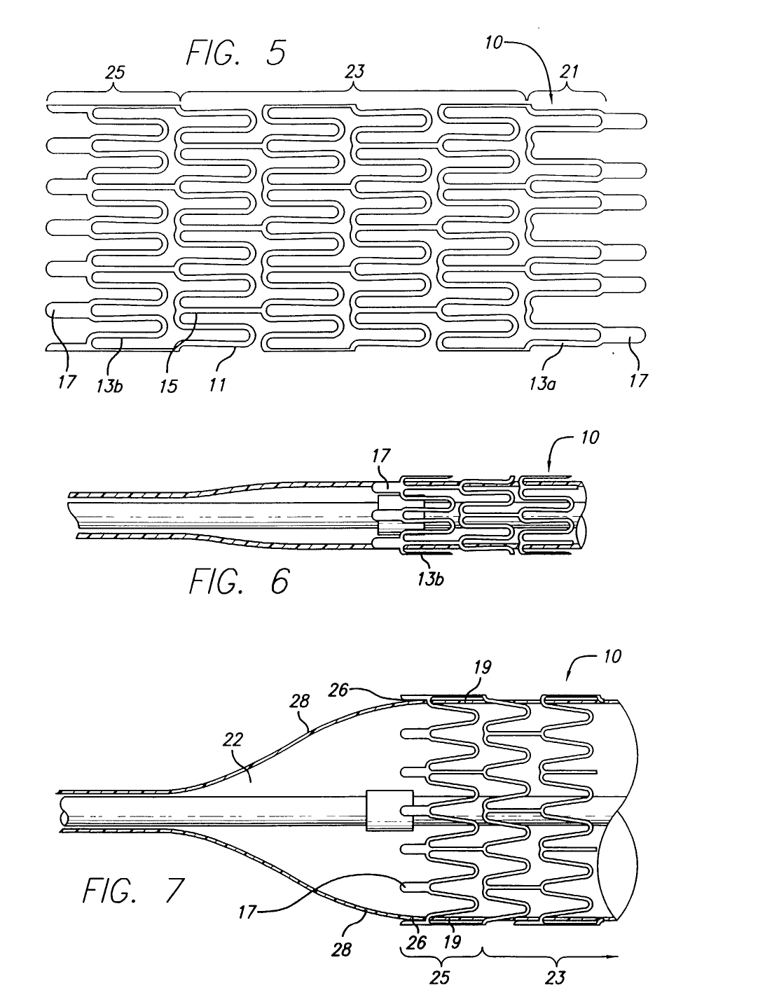

In one embodiment shown in FIG. 5, the stent 10 of the present invention has a series of flexible undulating cylindrical rings 11,13a,13b being expandable in a radial direction, with each of the rings having a first delivery diameter and a second implanted diameter and being aligned on a common longitudinal axis. At least one substantially linear link 15 attaches the distal ring 13a and the proximal ring 13b to the center rings 11 of the stent. The center rings may be connected similarly with substantially linear links. Preferably, each of the rings is formed of a metallic material. However, the stent of the present invention is not limited to the use of such metallic materials as non-metallic materials are also contemplated for use with the invention. The center rings 11 may be shorter along the longitudinal axis of the strut than the distal ring and proximal ring, both of which include the tabs 17.

The stent of FIGS. 1-5 is shown in FIG. 6 in its crimped form on an inflatable balloon. The distal ring 13a (not shown) and proximal ring 13b are formed with tabs 17 which extend away from the center section 23 and beyond the working length portion 19 of the balloon 22 to the transition portions 26 of the balloon in its expanded state shown in FIG. 7. The inclusion of the tabs makes the distal ring and the proximal ring longer along the longitudinal axis of the strut than the center rings. The balloon working length is generally defined as the length of the balloon consisting of a relatively constant outside diameter when the balloon is completely inflated. The transition portion of the balloon is that which exists between the taper portion 28 and the working length portion of the balloon.

In this embodiment, the tabs 17 expand with the expansion of the rings 13a,13b. It is therefore unnecessary to extend the balloon working length portion 19 to the tabs. The overlap of stent coverage area into the transition portion 26 of the balloon that the tabs provide enables the stent to have a higher coverage area than a conventional ring and link stent with a similarly sized balloon. One of the benefits of the increased coverage area is an increase in the area of vessel wall which can be exposed to a drug incorporated with the stent.

For current products made and sold by Advanced Cardiovascular Systems, Inc. of Santa Clara, Calif. such at the MULTI-LINK PENTA®, MULTI-LINK ZETA®, and MULTI-LINK VISION®, the length of each tab 17 can be approximately 0.4 mm in order to result in a mean stent-to-shoulder length (STS) of 0.0 mm for each end of the stent. The tabs can also be formed with a radial end, an extending loop and the surface area of the tabs can be varied to improve drug coverage. Furthermore, the width and length as well as the tabs can be varied in order to suitably conform to a vessel wall and lessen the chance of injury to the vessel. In terms of length, the tab length may be varied between 0.2 mm and 1 mm according to design requirements.

The stent also addresses the physician's concern of damaging the vessel wall during balloon inflation. Because the tabs 17 effectively extend the length of the stent further than the balloon working length portion 19 and into the transition portion 26, there is a reduced likelihood that the balloon transition portion will come into contact with the vessel wall and potentially cause injury.

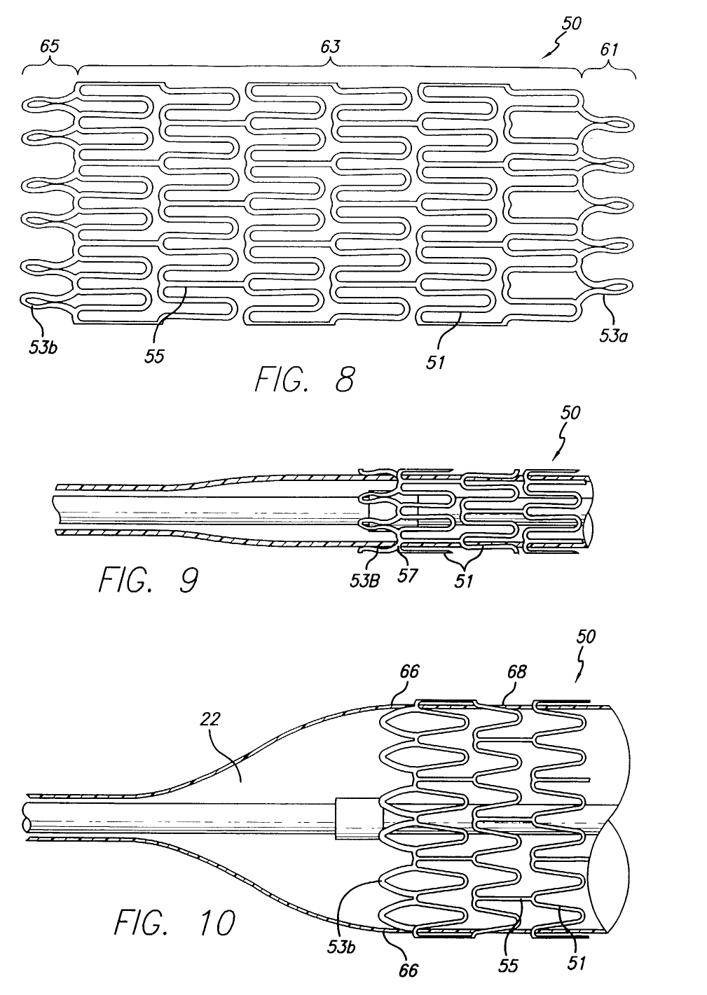

In another embodiment shown in FIG. 8, the stent 50 of the present invention includes a distal section 61, proximal section 65, and center section 63. Two sets of rings include a second set with a distal ring 53a located in the distal section and a proximal ring 53b located in the proximal section and a first set with a series of center rings 51 located in the center section. The cylindrical rings are expandable in a radial direction, with each of the rings having a first delivery diameter and a second implanted diameter and being aligned on a common longitudinal axis. At least one substantially linear link 55 connects adjacent center rings. Preferably, each of the rings is formed of a metallic material. However, the stent of the present invention is not limited to the use of such metallic materials as non-metallic materials are also contemplated for use with the invention. Converse to the stent shown in FIGS. 1-7, the stent shown in FIG. 8 incorporates center rings 51 which may be longer (along the longitudinal axis of the stent) than either or both the distal ring 53a and the proximal ring 53b.

The stent of FIG. 8 is shown in FIG. 9 in its crimped form on an inflatable balloon. The distal ring 53a (not shown) and proximal ring 53b are formed with a super-elastic material, preferably nickel-titanium (NiTi). The rings extend beyond the working length portion 68 of the balloon 72 to the transition portions 66 of the balloon in its expanded state shown in FIG. 10.

In this embodiment, the rings 53a,53b are connected directly to adjacent center rings 51. Because of their superelastic construction, the distal ring and proximal ring do not need to be expanded with the balloon, rather their expansion and contraction is directly related to the expansion and contraction of the center rings due to the direct connections 57 between the rings. It is therefore unnecessary to extend the balloon working length portion 68 of the balloon to these superelastically formed rings. The overlap that the rings provide enables the stent to have a higher coverage area than a conventional balloon expandable stent with a similarly sized balloon. One of the benefits of the increased coverage area is an increase in the area of the vessel which can be exposed to a drug incorporated with the stent.

For current products made and sold by Advanced Cardiovascular Systems, Inc. of Santa Clara, Calif. such at the MULTI-LINK PENTA®, MULTI-LINK ZETA®, and MULTI-LINK VISION®, the length of the distal ring 53a and proximal ring 53b can each be approximately 0.4 mm in order to result in a mean STS of 0.0 mm. The length as well as shape and pattern of the distal ring and proximal ring can be varied. For example, the ring length may range from 0.2 mm to 1 mm according to design requirements.

The stent also addresses the physician's concern of damaging the vessel wall during balloon 22 inflation. Because the rings 53a,53b extend outward of the balloon working length and into the transition portion there is a reduced likelihood that the balloon transition portion will come into contact with the vessel wall and potentially cause damage.

The stents of the present invention can be made in many ways. The preferred method of making the stent 10, shown in FIGS. 1-7 and the center section 63 of the stent 50 shown in FIGS. 8-10, is to cut tubing, such as stainless steel tubing, to remove portions of the tubing in the desired pattern for the stent, leaving relatively untouched the portions of the metallic tubing which are to form the stent. It is preferred to cut the tubing in the desired pattern by means of a machine-controlled laser, which is well known in the art.

The stent tubing may be made of suitable biocompatible material such as stainless steel, titanium, tungsten, tantalum, vanadium, cobalt chromium, gold, palladium, platinum, and iradium, and even high strength thermoplastic polymers. The stent diameters are very small, so the tubing from which it is made must necessarily also have a small diameter. For PCTA applications, typically the stent has an outer diameter on the order of about 1.65 mm (0.065 inch) in the unexpanded condition, the same outer diameter of the tubing from which it is made, and can be expanded to an outer diameter of 5.08 mm (0.2 inch) or more. The wall thickness of the tubing is about 0.076 mm (0.003 inch). For stents implanted in other body lumens, such as PTA applications, the dimensions of the tubing are correspondingly larger. While it is preferred that the stents be made from laser cut tubing, those skilled in the art will realize that the stent can be laser cut from a flat sheet and then rolled up in a cylindrical configuration with the longitudinal edges welded to form a cylindrical member.

In the instance when the stents are made from plastic, the implanted stent may have to be heated within the arterial site where the stents are expanded to facilitate the expansion of the stent. Once expanded, it would then be cooled to retain its expanded state. The stent may be conveniently heated by heating the fluid within the balloon or the balloon itself directly by a known method.

The distal ring 53a and proximal ring 53b of the stent 50 may be made of materials such as super-elastic (sometimes called pseudo-elastic) nickel-titanium (NiTi) alloys. In this case the rings would be formed full size but deformed (e.g. compressed) to a smaller diameter onto the balloon of the delivery catheter to facilitate intraluminal delivery to a desired intraluminal site. The stress induced by the deformation transforms the rings from an austenite phase to a martensite phase, and upon release of the force when the stent reaches the desired intraluminal location, allows the stent to expand due to the transformation back to the more stable austenite phase. Further details of how NiTi super-elastic alloys operate can be found in U.S. Pat. No. 4,665,906 (Jervis) and U.S. Pat. No. 5,067,957 (Jervis), incorporated herein by reference in their entirety. The NiTi alloy rings may be attached to the other rings through welding, bonding and other well known types of attachments.

The stent of the invention also can be coated with a drug or therapeutic agent. Further, it is well known that the stent (when made from a metal) may require a primer material coating such as a polymer to provide a substrate on which a drug or therapeutic agent is coated since some drugs and therapeutic agents do not readily adhere to a metallic surface. The drug or therapeutic agent can be combined with a coating or other medium used for controlled release rates of the drug or therapeutic agent. Examples of therapeutic agents or drugs that are suitable for use with the polymeric materials include sirolimus, everolimus, actinomycin D (ActD), taxol, paclitaxcl, or derivatives and analogs thereof. Examples of agents include other antiproliferative substances as well as antineoplastic, antiinflammatory, antiplatelet, anticoagulant, antifibrin, antithrombin, antimitotic, antibiotic, and antioxidant substances. Examples of antineoplastics include taxol (paclitaxel and docetaxel). Further examples of therapeutic drugs or agents that can be combined with the polymeric materials include antiplatelets, anticoagulants, antifibrins, antithrombins, and antiproliferatives. Examples of antiplatelets, anticoagulants, antifibrins, and antithrombins include, but are not limited to, sodium heparin, low molecular weight heparin, hirudin, argatroban, forskolin, vapiprost, prostacyclin and prostacyclin analogs, dextran, D-phe-pro-arg-chloromethylketone (synthetic antithrombin), dipyridamole, glycoprotein IIb/IIIa platelet membrane receptor antagonist, recombinant hirudin, thrombin inhibitor (available from Biogen located in Cambridge, Mass.), and 7E-3B® (an antiplatelet drug from Centocor located in Malvern, Pa.). Examples of antimitotic agents include methotrexate, azathioprine, vincristine, vinblastine, fluorouracil, adriamycin, and mutamycin. Examples of cytostatic or antiproliferative agents include angiopeptin (a somatostatin analog from Ibsen located in the United Kingdom), angiotensin converting enzyme inhibitors such as Captoprilg (available from Squibb located in New York, N.Y.), Cilazapril® (available from Hoffman-LaRoche located in Basel, Switzerland), or Lisinopril® (available from Merck located in Whitehouse Station, N.J.); calcium channel blockers (such as Nifedipine), colchicine, fibroblast growth factor (FGF) antagonists, fish oil (omega 3-fatty acid), histamine antagonists, Lovastatin® (an inhibitor of HMG-CoA reductase, a cholesterol lowering drug from Merck), methotrexate, monoclonal antibodies (such as PDGF receptors), nitroprusside, phosphodiesterase inhibitors, prostaglandin inhibitor (available from GlaxoSmithKline located in United Kingdom), Seramin (a PDGF antagonist), serotonin blockers, steroids, thioprotease inhibitors, triazolopyrimidine (a PDGF antagonist), and nitric oxide. Other therapeutic drugs or agents which may be appropriate include alpha-interferon, genetically engineered epithelial cells, and dexamethasone.

While the foregoing therapeutic agents have been used to prevent or treat restenosis, they are provided by way of example and are not meant to be limiting, since other therapeutic drugs may be developed which are equally applicable for use with the present invention. The treatment of diseases using the above therapeutic agents are known in the art. Furthermore, the calculation of dosages, dosage rates and appropriate duration of treatment are previously known in the art.

While the invention has been illustrated and described herein in terms of its use as intravascular stents, it will be apparent to those skilled in the art that the stents can be used in other instances in all vessels in the body. Since the stents of the present invention have the novel features of enhanced safety and coverage area due to the extensions and superelastic rings, they are particularly well suited for implantation in almost any vessel where such devices are used. This feature, coupled with limited longitudinal contraction of the stent when radially expanded, provides a highly desirable support member for all vessels in the body. Other modifications and improvements may be made without departing from the scope of the invention.

What is claimed:

1. An intravascular stent, comprising:
a plurality of a first set of metallic cylindrical rings and a plurality of a second set of metallic cylindrical rings, each set of cylindrical rings being radially expandable, longitudinally aligned, and each with a first delivery diameter and a second implanted diameter;
a plurality of substantially linear metallic links connecting a plurality of adjacent cylindrical rings;
wherein the stent includes a distal section with one distal ring, a center section with a plurality of rings, and a proximal section with one proximal ring;
wherein the first set of rings are formed with a first longitudinal length and the second set of rings are formed with a second, relatively smaller longitudinal length.

2. The stent of claim 1, wherein the first set of rings are located in the proximal ring and the distal ring and the second set of rings are located within the center section of the stent.

3. The stent of claim 2, wherein the distal ring and proximal ring are formed with peaks and valleys comprising U- and W-shaped undulations and with a plurality of tabs formed on a plurality of the peaks and valleys, the tabs extending away from the center section and along a longitudinal axis of the stent.

4. The stent of claim 3, wherein a plurality of the tabs are formed on the peaks of the distal ring and wherein a plurality of the tabs are formed on the valleys of the proximal ring.

5. The stent of claim 4, wherein the tabs are each between 0.2 mm and 1 mm long.

6. The stent of claim 5, wherein the tabs are each approximately 0.4 mm long.

7. The stent of claim 4, wherein the tabs are formed with radiused ends.

8. The stent of claim 1, wherein the first set of rings are located within the center section of the stent and wherein the second set of rings comprise the proximal ring and the distal ring.

9. The stent of claim 8, wherein the proximal ring is directly connected to an adjacent ring within the center section and wherein the distal ring is directly connected to an adjacent ring within the center section.

10. The stent of claim 9, wherein the material forming the second set of cylindrical rings embodies shape memory characteristics.

11. The stent of claim 10, wherein the shape memory material is superelastic material.

12. The stent of claim 11, wherein the superelastic material is nickel-titanium.

13. The stent of claim 12, wherein the proximal ring and distal ring are each between 0.2 mm and 1 mm long.

14. The stent of claim 13, wherein the proximal ring and distal ring are each approximately 0.4 mm long.

15. The stent of claim 1, wherein the stent is biocompatible.

16. The stent of claim 1, wherein the stent is non-biodegradable.

17. The stent of claim 1, wherein the stent includes a material therein to enhance the radiopacity of the stent.

18. The stent of claim 1, wherein the stent is formed with substantially translucent members.

19. The stent of claim 1, wherein the stent may be expanded by force.

20. The stent of claim 1, wherein the metallic materials forming the cylindrical rings and links is taken from the group of metals consisting of stainless steel, titanium, tungsten, tantalum, vandium, nickel-titanium, cobalt-chromium, gold, palladium, platinum and iridium.

21. The stent of claim 1, wherein at least a portion of the stent is coated with a therapeutic agent or drug.

22. An intravascular stent, comprising:
a plurality of a first set of cylindrical rings and a plurality of a second set of cylindrical rings, each set of cylindrical rings being radially expandable, longitudinally aligned, and each with a first delivery diameter and a second implanted diameter;
a plurality of links connecting a plurality of adjacent cylindrical rings;
wherein the stent includes a distal section, a center section, and a proximal section;
wherein the first set of rings are located within the center section and balloon expandable and wherein the second set of rings are located within the distal section and proximal section and formed from a self-expanding material.

23. An intravascular stent, comprising:
a plurality of a first set of cylindrical rings and a plurality of a second set of cylindrical rings, each set of cylindrical rings being radially expandable, longitudinally aligned, and each with a first delivery diameter and a second implanted diameter;
a plurality of links connecting a plurality of adjacent cylindrical rings;
wherein the stent includes a distal section, a center section, and a proximal section;
wherein the first set of rings are located within the distal section and the proximal section and formed with a plurality of tabs extending away from the center section and wherein the second set of rings are located within the center section and formed with a plurality of undulations.

24. The stent of claim 1, wherein the stent is configured to be mounted on a balloon with a balloon working length and wherein the stent has a length approximately equal to the balloon working length.
